SEQUENCE LISTING (7 sequences)

SEQ ID NO: 1 - DNA, 22 bp, Artificial Sequence
  Synthetic
atgaactacccgctaacgctgg

SEQ ID NO: 2 - DNA, 25 bp, Artificial Sequence
  Synthetic
ctagaacgtggtgagagaggtggca

SEQ ID NO: 3 - DNA, 20 bp, Artificial Sequence
  Synthetic
taatacgactcactataggg

SEQ ID NO: 4 - DNA, 19 bp, Artificial Sequence
  Synthetic
gaaggtgaaggtcggagtc

SEQ ID NO: 5 - DNA, 20 bp, Artificial Sequence
  Synthetic
gaagatggtgatgggatttc

SEQ ID NO: 6 - DNA, 19 bp, Artificial Sequence
  Synthetic
ccttgaaggaggccatgag

SEQ ID NO: 7 - DNA, 20 bp, Artificial Sequence
  Synthetic
taacgctggaaatggacctc

(19) United States
(12) Patent Application Publication
(10) Pub. No.: US 2020/0215117 A1
(43) Pub. Date: Jul. 9, 2020

(54) MESENCHYMAL STEM CELL OVER-EXPRESSING CXCR5, PREPARATION METHOD AND USE THEREOF

(71) Applicant: SUN YAT-SEN UNIVERSITY, Guangzhou (CN)

(72) Inventors: Peng Xiang, Guangzhou (CN); Xiaoran Zhang, Guangzhou (CN); Xiaoyong Chen, Guangzhou (CN)

(21) Appl. No.: 16/821,061

(22) Filed: Mar. 17, 2020

Related U.S. Application Data
(63) Continuation of application No. PCT/CN2017/102263, filed on Sep. 19, 2017.

(51) Int. Cl. A61K 35/28; C12N 5/0775; C12Q 1/686; C12Q 1/6853

(52) U.S. Cl. CPC A61K 35/28; C12N 5/0663; C12N 2510/00; C12Q 1/6853; C12Q 1/686

(57) ABSTRACT

The disclosure provides a mesenchymal stem cell (MSC) over-expressing CXCR5, preparation method and use thereof. Overexpression of CXCR5 can allow directed migration of MSC^{CXCR5} to an inflammation area in vivo so as to play a role in immunomodulation but not make MSC^{CXCR5} randomly scattered at various parts in a body. Treatment of diseases using the mesenchymal stem cells will have more targeting and effectiveness, and will effectively improve the treatment effect of MSC.

### CROSS-REFERENCE TO RELATED APPLICATION

This application is a continuation application of PCT Application No. PCT/CN2017/102263 filed on Sep. 19, 2017, the entire content of which is incorporated herein by reference.

### REFERENCE TO SEQUENCE LISTING

The Sequence Listing is submitted as an ASCII formatted text file via EFS-Web, with a file name of “Sequence_listing.TXT”, a creation date of Mar. 17, 2020, and a size of 1,629 bytes. The Sequence Listing filed via EFS-Web is part of the specification and is incorporated in its entirety by reference herein.

### TECHNICAL FIELD

The disclosure relates to mesenchymal stem cells (MSC) over-expressing CXCR5, preparation method and use thereof.

### BACKGROUND

Mesenchymal stem cells (MSC) are non-hematopoietic stem cells (Friedenstein, A. J., et al, 1974) that are first found in bone marrow, which participate in forming the hematopoietic microenvironment of bone marrow and have an obvious support effect on proliferation and differentiation of hematopoietic stem cells (Mendez-Ferrer, S., et al., 2010). MSC is widely distributed in various tissues and organs of the whole body, which, except for bone marrow, can also be isolated from umbilical cord, umbilical blood, gingiva, skeletal muscles and the like. MSC also has an ability of differentiating into osteoblasts, chondrocytes, adipocytes, cardiomyocytes and the like. MSC is lack of specific markers, mainly expresses mesenchymal markers such as CD29, CD44, CD73, CD90, CD105 and CD166, and does not express hematopoiesis-related markers such as CD11b, CD14, CD19, CD34 and CD45; does not express or low express HLA-I molecules, and does not express HLA-II molecules.

In recent years, more and more attentions are paid to specific immunomodulatory effect of MSC. In vitro experiments show that MSC can inhibit the proliferation of T lymphocytes in specifically stimulated mixed lymphocyte reaction (MLR) or induced by non-specific mitogen phytolectin (PHA) stimulation (Di Nicola, M., et al., 2002). MSC can induce the amplification of regulatory T cells (Treg) and inhibit the cytotoxicity function of killer T cells (Aggarwal, S., et al., 2005). MSC can influence the activation, proliferation and chemotaxis of B cells and generation of antibodies thereof (Corcione, A., et al., 2006). MSC can inhibit the generation and proliferation of DC and block the maturation and differentiation of DC, thereby weakening the antigen presenting ability of DC (Beyth, S., et al., 2005). MSC can strongly inhibit the proliferation of NK cells activated by IL-2 as well as secretion and killing function of cytokines. The mechanism of MSC regulating immune cells has not been completely understood and its immunomodulatory effect is not limited by MHC, has a direct contact action mode and can secrete soluble factors to take the effects of transforming growth factor-β (TGF-β), interleukin-10 (IL-10), prostaglandin E2 (PGE2), hepatocyte growth factor (HGF), tumor necrosis factor-α stimulated gene-6 (TSG-6) and the like (Gebler, A., O. Zabel and B. Seliger, 2012). MSC is easy to isolate and amplify and has low immunogenicity and has the functions of paracrine and immunomodulation, so it has a broad application prospect in the aspect of cell therapy. At present, there are more than 600 clinical trials for mesenchymal stem cells (April 2015, clinicaltrials.gov), and indications include autoimmune diseases, transplantation rejection, bone/cartilage diseases, cardiovascular diseases, nervous system diseases and the like. The effectiveness of the applicant's early work for the treatment of chronic graft-versus-host diseases has confirmed the immunomodulatory function of MSC to diseases(Peng, Y., et al., 2015). It is reported that MSC can also be used for treating multiple sclerosis (Li, J. F., et al., 2014), ulcerative colitis (Duijvestein, M., et al., 2010), diabetes (Holmes, et al., 2014) and the like.

However, we find that in clinical treatment, the effect is sometimes not ideal. Some researchers believe that although MSC has good immunomodulatory function in vitro, for example, is capable of effectively inhibiting the proliferation of T cells and the like, the intravenously infused MSC can not completely treat mouse cGVHD (Sudres M. et al, 2006); some other researchers suggest that there are few MSCs to gather in injured parts when liver injury is treated and believe that it possibly is the main reason that the treatment effect of MSC is limited (Gao J. etc, 2001); via tracking, some researchers find that after entering the body, MSCs are scattered in various organs, such as lung, liver and bone marrow (Paul Lin. et al, 2013), which is equivalent to fact that MSC is “diluted” in the body, there is no considerable quantity of MSCs that really reach the key parts to take effects, and thus the immunomodulatory function will be greatly weakened. Therefore, the main problem faced by MSC in clinical treatment is disordered distribution of MSC in the body after infusion.

Chemokines are crucial in the development and progression of diseases, and there are many chemokine receptors such as CCR1, CCR4, CCR6, CCR7, CCR9, CCR10, CXCR4, CXCR5, CXCR6 and CX3CR expressed on the surface of MSC. Not only are these receptors selves express low in expression quantity, but also MSC hardly expresses these receptors after amplification and passage (Sarkar, D., et al., 2011). However, the content of MSC in bone marrow only accounts for 0.001-0.01%. The quantity of primary MSC isolated from bone marrow donated once by a healthy donor is limited. Generally, the number of cells infused once for treating cGVHD patients is 0.4-9×10⁶ cells/kg weight, and the number of cells required for once treatment is about 0.2-4.5×10⁸. Therefore, in-vitro large-scale amplification of MSC is a basic method for reaching the number of cells required for clinical application at present. This may cause the instability of treatment due to the loss of MSC receptor expression.

At present, many researchers take improvement of targeting of MSC treatment as their research directions: for example, CXCR4 is overexpressed using a virus transfection method to promote the chemotaxis of MSC into brain injured parts (Wang, Z., et al., 2015); MSC is pretreated with IGF-1 to increase the expression of CXCR4 on MSC (Xinaris, C., et al., 2013); by increasing adhesion molecules PSGL-1 and SLeX, the adhesion ability of MSC to inflammatory endothelial cells is enhanced (Levy, O., et al., 2013); transformation of CD44 molecules on MSC into E-selectin/L-selectin increases the chemotaxis of MSC to bone marrow (Sackstein, R., et al., 2013), etc.

The chemokine CXCL13 plays an important role in inflammatory response, for example, mRNA high expression of CXCL13 is detected in inflammatory synovium fluid of autoimmune synovitis (William H, et al., 2013); high expression of chemokine CXCL13 in the inflammatory part is also found in the disease model of mouse ileitis (A Viejo-Borbolla, et al, 2010); in the neural Lyme disease, the level of CXCL13 in cerebrospinal fluid can even be used as an index of high sensitivity in acute attack (Katie Kingwell, 2011). We also find that in the mouse contact hypersensitivity (CHS) model, CXCL13 gradually rises with the development of local ear inflammation, and this model has rapid onset and limited inflammatory response, which is conducive to observing results.

Accordingly, we construct the plasmid of receptor CXCR5 corresponding to chemokine utilizing this feature. By utilizing the mRNA modification method, MSC expresses receptor CXCR5, and MSC over-expressing CXCR5 is infused at the peak of the disease. Through observation, we find that MSC over-expressing CXCR5 is significant in treatment effect and can directionally migrate to lesion sites when in the body, thereby greatly improving the efficiency of MSC exerting the ability of immunomodulation in vivo. In addition, we establish an inflammatory bowel disease (IBD) model to explore whether MSC over-expressing CXCR5 can also play a similar role in other disease models.

### SUMMARY

In the present disclosure, by transferring the mRNA of CXCR5 into MSC, MSC overexpresses the receptor CXCR5, MSC can directionally migrate to the lesion sites when being intravenously infused into the body, rather than being scattered randomly in various parts of the body, thereby greatly improving the efficiency of MSC exerting the ability of immunomodulation in vivo.

In order to achieve the above object, the technical solution adopted by the disclosure is to provide a mesenchymal stem cell over-expressing CXCR5.

The disclosure provides use of the above MSC in preparation of a drug for treating inflammatory diseases. Preferably, the inflammatory disease sites contain chemokine CXCL13.

The disclosure provides the use of the above MSC in preparation of a drug for treating one of transplant rejection, multiple sclerosis, ulcerative colitis, diabetes and ileitis.

The disclosure provides a drug comprising the above MSC.

The disclosure provides a preparation method of the above MSC, the preparation method comprising: transferring mRNA of CXCR5 into MSC to obtain MSC over-expressing CXCR5.

Preferably, the transfer manner is an electroporation transfection method.

Preferably, the preparation method of the mRNA of CXCR5 comprises the following steps:

(1) collecting and culturing mononuclear cells in peripheral blood from a healthy individual to obtain mononuclear cells of peripheral blood;

(2) extracting RNA of mononuclear cells of peripheral blood;

(3) reverse transcription;

(4) PCR reaction and recovering CXCR5 cDNA fragments;

(5) constructing DNA fragments for transcription of CXCR5 mDNA;

(6) mRNA in vitro transcription.

Preferably, primers used in PCR reaction in step (4) are as follows:

upstream primer:
(SEQ ID NO. 1)
ATGAACTACCCGCTAACGCTGG
downstream primer:
(SEQ ID NO. 2)
CTAGAACGTGGTGAGAGAGGTGGCA.

Preferably, the DNA fragment in step (5) is obtained by linking the CXCR5 cDNA fragment in step (4) to plasmids.

Preferably, the DNA fragment in step (5) is obtained by performing PCR with the CXCR5 cDNA fragment in step (4) as a template and a CXCR5 cDNA upstream primer with a promoter and a CXCR5 cDNA downstream primer as upstream and downstream primers respectively.

The disclosure has the beneficial effects that by constructing a plasmid over-expressing CXCR5 and utilizing mRNA modification manner of CXCR5, MSC has more efficiency and safety except for the characteristic of overexpressing CXCR5 relative to chronic virus infection and other ways. Different from ordinary MSC, on the one hand, MSC^{CXCR5} can quickly and effectively migrate to lesion sites to exert the ability of immunomodulation but not is unintentionally and randomly scattered in various parts of the body; on the other hand, the problems that MSC itself is low in expression quantity, and MSC hardly expresses the CXCR5 receptor after amplification and passage are solved. More specifically, the disclosure overexpresses CXCR5 on the basis of not influencing the phenotype, differentiation ability and immunoregulation ability of MSC, so that MSC^{CXCR5} can directionally migrate to an inflammation parts in the body, thereby more effectively exerting the object of targeted treatment. Particularly, treatment methods for autoimmune diseases are optimized. By modifying MSC with CXCR5 genes, treatment has better targeting and effective, and the treatment effect of MSC can be more significantly improved.

### BRIEF DESCRIPTION OF THE DRAWINGS

FIG. 1: mRNA and protein levels of CXCR5 expressed by MSC^{CXCR5} and MSC^{EGFP};

FIG. 2: a graph illustrating changes in mRNA of CXCL13 of the right ear (sensitized) after sensitization over time;

FIG. 3: modelling and treatment pattern of CHS mice;

FIG. 4: a line chart of edema degree of mouse ears after treatment;

FIG. 5: staining results of lesion site sections;

FIG. 6: a histogram illustrating MPO activity of ears of CHS mice;

FIG. 7: expression level of inflammatory factors in local tissues;

FIG. 8: inflammatory response-colon length resulting from significantly reducing IBD by MSC^{CXCR5};

FIG. 9: inflammatory response-mouse weight resulting from significantly reducing IBD by MSC^{CXCR5};

FIG. 10: expression level of inflammatory factors in local tissues;

FIG. 11: staining results of lesion site sections.

### DESCRIPTION OF THE EMBODIMENTS

For making the technical solution and advantages of the disclosure more clearly, the disclosure will be described in detail in combination with drawings and embodiments.

In the following examples, experimental methods without specific conditions are generally carried out in accordance with conventional conditions or conditions recommended by the manufacturer. The “room temperature” in the examples refers to the temperature of a operating room for experiments, generally 25° C.

Unless otherwise specified, all the reagents used in the following examples can be purchased from chemical or biological reagent stores or suppliers; the used instruments are also conventional instruments in the art.

### EXAMPLE 1

1. MSC Culture and Phenotype Identification

20 ml of bone marrow from healthy volunteers was taken and diluted with 1×PBS in a ratio of 1:1. Mononuclear cells were isolated from bone marrow with Ficoll-Paque lymph separating medium by density gradient centrifugation (2000 rpm, 30 min). The collected mononuclear cells were seeded into a 75 cm² culture flask for culture at a density of 1×10⁵/cm². After culture for 3 days with L-DMEM medium under the conditions of 37° C. and 5% CO₂, the suspension cells were removed and the remaining cells continued to be cultured after fluid was changed. After the cells grew to 80% density, the culture medium was sucked off, the cells were washed twice with PBS, digested for 1-2 min with 0.125% trypsin and passaged in a passage ratio of 1:3. MSC was isolated from bone marrow donated by healthy donors. The isolation, amplification, cryopreservation and resuscitation of clinical MSC were carried out in accordance with GMP (good manufacturing practice) standards. Under the inverted microscope, the growth and morphological characteristics of primary and passaged cells were observed every day and recorded by photograph. The MSC cultured in vitro was taken and digested into a single cell suspension, the single cell suspension was washed once with PBS (pH 7.4) containing 0.1% BSA+0.05% NaN₃, and the supernatant was discarded. The cell density was adjusted to 10⁶/ml in a flow tube. The MSC was labeled with flow antibodies CD29, CD34, CD44, CD45, CD73, CD90, CD105 and CD166. The MSC and the antibodies were sufficiently oscillated and evenly mixed and incubated at 4° C. for 30 min in the dark, and then the incubated mixture was washed twice with PBS (pH 0.1% BSA+0.05% NaN₃) (7.4) twice to remove the surplus antibodies; the supernatant was discarded and the cells were resuspended with 200 ul of 1% PFA. MSC cell phenotypes (CD29⁺, CD34⁻, CD44⁺, CD45⁻, CD73₊, CD90⁺, CD105⁺and CD166⁺) were detected using Flow cytometry, proving that in vitro culture has no influence on MSC cell phenotypes.

P2 cells were transferred into six-well plate and grew to about 60% for later use.

2. Obtaining of Peripheral Blood Mononuclear Cells

20 ml of fresh peripheral blood was collected from healthy volunteers, diluted with 1×PBS at 1:1, and then isolated with a Ficoll-Paque lymph separating medium by density gradient centrifugation. All the mononuclear cells on the white membrane layer were collected and then diluted with sterile PBS in a ratio of 1:4 and centrifuged for 10 min at 2000 rpm. Then the supernatant was discarded. The resulting cells were washed twice by adding sufficient PBS. Cells were suspended with a RPMI-1640 complete medium, so as to obtain human peripheral blood mononuclear cells (PBMC).

3. Extraction of Peripheral Blood Mononuclear Cell RNA with Trizol Method

The obtained human peripheral blood mononuclear cells (PBMCs) were added to 1 ml of Trizol solution and then evenly mixed by blowing, so that the cells were fully lysed and allowed to stand for 5 min; 200 μl of chloroform was added, the mixture was vigorously oscillated and evenly mixed for 20 s so that water phase and organic phase were fully contacted, and allowed to stand for 15 min at room temperature; the obtained mixture was centrifuged at 4° C. at 12000 rpm for 15 min. Then the mixture was divided into three layers, wherein RNA was in the upper water phase, and carefully transferred to another new RNase free EP tube; 0.5 ml of isopropanol was added, and mixed gently and thoroughly, and allowed to stand for 10 min at room temperature to precipitate RNA; the mixture was centrifuged for 10 min at 12000 rpm at 4° C., RNA precipitate was collected, and the supernatant was discarded; the tube wall was washed twice with 75% ethanol, and dried in the air on an ultra-clean table; 50 μl of DEPC water was added to dissolve the precipitate, and the concentration was measured with a Nanodrop ultra-micro spectrophotometer.

4. Reverse Transcription

Removal of genome DNA: RNA (1 μg)+DNase I (1 μl)+Buffer DNase I with MgCl₁₂ (1 μl)+DEPC water (totaling 10 μl system) were incubated for 30 min; EDTA (1 μl) was added to incubate for 10 min at 65° C.; Oligo (dT) (1 μl) was added to incubate for 10 min at 65° C.; finally, 5×Reaction Buffer (5 μl), RNase-Ribonuclease Inhibitor (1 μl), 10 mM dNTP Mix (2 μl), M-MLV RT (1 μl) and RNase Free Water (totaling 25 μl system) were added to be incubated for 60 min at 42° C., and the cDNA product was collected.

5. PCR Reaction and Recovery of CXCR5 cDNA Fragments

PCR reaction: 10 μl of 2×Star mix (containing Taq DNA Polymerase, dNTPs, Mg²⁺, reaction buffer and stabilizer, etc.), 7 μl of DEPC water, 1 μl of upstream primer, 1 μl of downstream primer, 1 μl of cDNA, total system 20 μl; CXCR5 fragment 1119 bp. Gel recovery: a target fragment strip was cut with a sharp scalpel, and PCR target gene CXCR5 fragments were recovered using the agarose gel DNA recovery kit

Where, primer sequences used when the target gene CXCR5 fragments were obtained through PCR were as follows:

upstream primer:
(SEQ ID No: 1)
ATGAACTACCCGCTAACGCTGG,
downstream primer:
(SEQ ID No: 2)
CTAGAACGTGGTGAGAGAGGTGGCA

6. In Vitro mRNA Transcription

CXCR5 cDNA was linked to the pCM-T7 plasmid with a seamless cloning method (Yeasen, 10912) to construct a template pCM-T7-CXCR5 plasmid for in vitro transcription; or the T7 promoter sequence and the 5′ sequence of CXCR5 cDNA were used as the upstream primer, the 3′ sequence of CXCR5 cDNA was used as the downstream primer (T7 promoter: TAATACGACTCACTATAGGG (SEQ ID No: 3)), CXCR5 CDNA is the template, and PCR amplification product is the template of transcription in vitro. The constructed pCM-T7-CXCR5 plasmid was linearized (Hind III-F enzyme in NEB cutsmart buffer for 6 hours at 37° C.) and then purified for recovery; or the PCR product was purified for recovery (Qiagen) and dissolved with RNase free water.

In vitro transcription system: 500ng−1 μg of linearized plasmid (or T7-CXCR5 PCR product) was used as the template, 10×T7 reaction buffer, T7 2×NTP/ARCA, T7 enzyme Mix (3 hours, 37° C., Ambion Kit: AM1345). 30 μl of system transcription products were purified by Ambion kit: AM1908, and the final yield was 15-20 ug.

7. Modification of MSC with Target mRNA (Target mRNA was Transferred into MSC)

When hMSC cells were cultured to 80-90% confluence, they were digested with 0.125% trypsin, washed with PBS for three times, collected and counted as 10⁶. The cells were then resuspended with 500 μl of MSC serum-free medium mixed with 5 μg of RNA product. The resuspended cells were added to the biorad 0.4 cm spacing shock cup (300V, 300 μF) for shock once. The shocked cells were placed at room temperature for 3 minutes, and then added to the 6-well plate to be further cultured for 24 hours.

8. Detection of Expression of CXCR5mRNA and Protein

After 24 hours, two groups of mRNA modified cells were collected, sorted and purified by flow cytometry to obtain a transgenic cell line and amplify it;

Real-time quantitative PCR (RT-PCR) was used to detect the content of mRNA of CXCR5-exoressing MSC which was infected:

\textcircled{1} RNA extraction and reverse transcription: steps refer to steps 3 and 4 in example 1;

\textcircled{2} PCR amplification reaction system:

| | | |
|---|---|---|
| cDNA | 1 | μl; |
| SYBR mix | 10 | μl; |
| 10 μM upstream primer | 1 | μl; |
| 10 μM downstream primer | 1 | μL; |
| ddH₂O | 7 | μl; |
| Total system | 20 | μl; |

Reaction conditions: 95° C., 10 min; 3-step method, 40 cycles: 95° C., 15 s, 60° C., 30 s, 72° C., 15 s; dissolution curve: 55° C.-95° C., read once per minute.

Wherein, the primers used in the PCR amplification reaction system are as follows:

GAPDH:
upstream primer:
(SEQ ID No: 4)
GAAGGTGAAGGTCGGAGTC
downstream primer:
(SEQ ID No: 5)
GAAGATGGTGATGGGATTTC
CXCR5:
upstream primer:
(SEQ ID No: 6)
CCTTGAAGGAGGCCATGAG
downstream primer:
(SEQ ID No: 7)
TAACGCTGGAAATGGACCTC.

Detection of protein level of CXCR5 expressed by target mRNA modified MSC with Western Blot

\textcircled{1} Extraction of protein: MSC^{CXCR5} over-expressing CXCR5 in culture medium and control group MSC (MSC^{EGFP}) were taken and placed on ice, the culture solution was removed, the cells were washed twice with pre-cooled PBS, then 1×SDS sampling buffer containing 5% DTT (62.5 mM Tris-HCl (pH 6.8), 2% (w/v) SDS, 10% glycorol, 50 mM DTT, 0.1% (w/v) Bromophenol blue) was added, and 1 ml pipette gun (about 10 times) was used for blowing back and forth to fully lyse the cells. After blowing, the liquid was sucked and put into 1.5 ml Eppendorf centrifuge tube, and broken 3 times at 4° C. for 1 s each time under the ultrasonic condition; boiled at 100° C. for 5 min, cooled at 4° C. and centrifuged at 4° C. for 5 min at 15000 r, and the cells were prepared for electrophoresis or stored at −80° C. for later use.

\textcircled{2} Isolation of samples with gel electrophoresis: denatured polyacrylamide gel (SDS-PAGE): 4.1 mL of isolated gel was added and subjected to layer seal with deionized water. After an obvious interface appears, the water seal layer was removed and the prepared spacer gel was added to the top of the short glass block. A comb was inserted, when the gel surface was in irregular shape, it indicates that the gel has polymerized well, and the comb can be pulled out and loading was carried out.

In order, 15 μL of sample boiled at 100° C. for 5 minutes was added in each lane, and subjected to electrophoresis for about 45 min in SDS electrophoresis buffer at 120V constant pressure.

\textcircled{3} Membrane transfer: while electrophoresis, materials such as sponge, filter paper and PVDF membrane, required for membrane transfer were soaked in membrane transfer buffer (25 mm Tris base, 0.2 M glycine, 20% methanol pH 8.5). After electrophoresis was ended, the gel was removed and the spacer gel on the upper layer was removed. The gel was put in the membrane transfer liquid to be balanced for 15-30 minutes to remove SDS attached to the surface of the gel. Then the transfer membrane sandwich box started to install from negative pole to positive pole in a sequence of sponge, filter paper, gel, PVDF film, a layer of filter paper and sponge. After fixing, the sandwich box was put in the transfer tank, and the PVDF membrane face was toward the positive pole. The sandwich box and the ice box were placed in the transfer tank, 600 ml transfer buffer was injected, and 200 mA constant current was maintained for 2 h.

\textcircled{4} Antigen and antibody reaction:

At the end of membrane transfer, the PVDF membrane was removed, washed in 25 mL of TBS (50 mm Tris HCl ph7.4150 mm NaCl) for 10 min, then transferred to 20 ml closure solution [1×TBST (0.05% Tween-20 in TBS) containing 5% skimmed milk], shaken and sealed for 1 h at room temperature. Corresponding primary antibody dilution solution diluted by 5% skimmed milk was added. The above mixture was oscillated for overnight at 4° C. On the next day, the membrane was washed 3 times with 1×TBST, each time for 5 minutes, and then 15 ml of HRP labeled secondary antibody (1:2000) diluted with closure solution and labeled with horseradish peroxidase (HRP) was added to oscillation for 1 h at room temperature. Then the membrane was washed 3 times with 1×TBST, 10 minutes each time, and developed and fixed in the darkroom: ECL Kit A and B solution was taken and prepared into work solution to be uniformly applied to the surface of the PVDF membrane. After incubation for 1 min, the reaction residue solution on the surface of the membrane was removed as much as possible. The membrane was fixed with a plastic preservative film in an X-ray box, the X-ray film was put for moderate exposure, the X-ray film was taken out and reacted in the developing solution for 1 minute, the developed film was rinsed in water for several times, reacted in the fixing solution for 1 min, and then wash with water and aired.

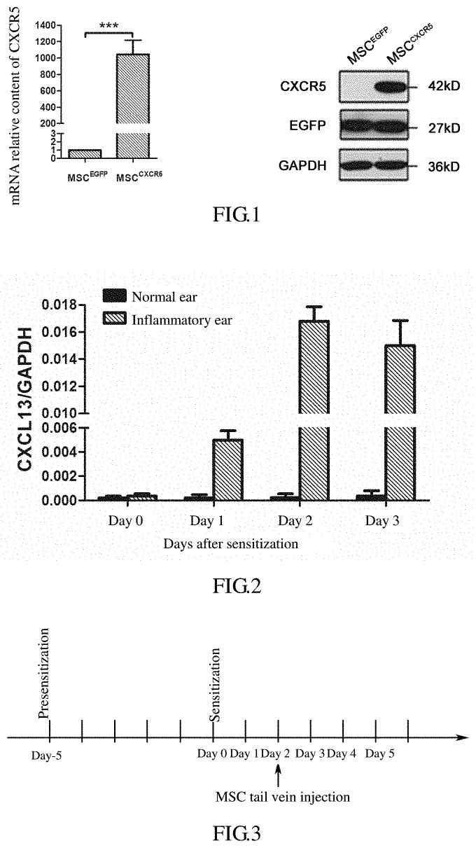

The results can be seen in FIG. 1, MSC^{CXCR5} expresses protein CXCR5, MSC^{EGFP} low expresses CXCR5, and both of them express green fluorescent protein EGFP. In order to detect the influence of CXCR5 on the directed migration ability of MSC, we construct the plasmid over-expressing CXCR5 and the control group plasmid. MSC over-expressing CXCR5 was recorded as MSC^{CXCR5} and control group MSC was recorded as MSC^{EGFP}. Compared with MSC^{EGFP}, MSCC^{CXCR5} cells significantly increased the mRNA and protein levels of CXCR5 (FIG. 1).

EXAMPLE 2: DNFB-Induced Mouse ContactCONTACT HypersensitiveReaction

DNFB (2,4-dinitro-1-fluorobenzene)-induced mouse contact hypersensitive reaction model was constructed as follows:

1. 6˜8-week male BALB/c mice, 16˜18 g, feeding in SPF environment

2. Preparation of reagents

\textcircled{1} DNFB pre-sensitization solution

Mixed solution (acetone: olive oil 4:1) was prepared and evenly and acutely oscillated

Preparation of pre-sensitization mixed solution: 0.5% DNFB mixed solution, namely, pre-sensitization mixed solution, was prepared with mixed solution.

\textcircled{2} DNFB sensitization solution: 0.2% DNFB mixed solution, namely, pre-sensitization mixed solution, was prepared with mixed solution.

3. Pre-sensitization: on the 1d, a 1.5×1.5 cm region was scraped from the skin near the head of the mouse's back with an electric razor. 20 μl of 0.5% DNFB pre-sensitization mixed solution was smeared to the scraped skin. After treatment, the mice were fed regularly.

4. Sensitization: on the d5, the ears of mice were smeared with 0.2% DNFB mixed solution, and 10 μl on both sides of the ears.

5. Animals were divided into four groups, 8 animals per group

\textcircled{1} Control group;

\textcircled{2} Model (CHS) group;

\textcircled{3} MSC^{EGFP} (CHS+MSC^{EGFP}) treatment group;

\textcircled{4} MSC^{CXCR5} (CHS+MSC^{CXCR5})treatment group.

On the 2d after modeling, MSC^{EGFP} was injected intravenously in CHS+MSC^{EGFP} treatment group, with 1×10⁶ cells/animal;

MSCC^{CXCR5} was injected intravenously in CHS+MSC^{CXCR5} treatment group, with 1×10⁶ cells/animal.

6. The thicknesses of mouse ears were measured with a micrometer and recorded on the d1, d2, d3, d4 and d5 after treatment.

The results show that:

1. After sensitization, the mRNA of chemokine CXCL13 in the right ear (sensitized) of mice is increased continuously after inflammation (FIG. 2), the thickness is gradually increased, and the degree of edema is deepened.

2. Referring to FIG. 3, Day-5, 20 μl of 0.5% DNFB pre-sensitization mixed solution is applied to the back; Day 0, the right ear of mice is sensitized; on the 2d after sensitization, namely, Day 2, when the secretion of CXCL13 increases, mice are intravenously injected with MSC via tails for treatment:

MSC^{EGFP} was injected intravenously for CHS+MSC^{EGFP} treatment group, with 1×10⁶ cells/animal;

MSC^{CXCR5} was injected intravenously for CHS+MSC^{CXCR5} treatment group, with 1×10⁶ cells/animal;

PBS was injected for control group.

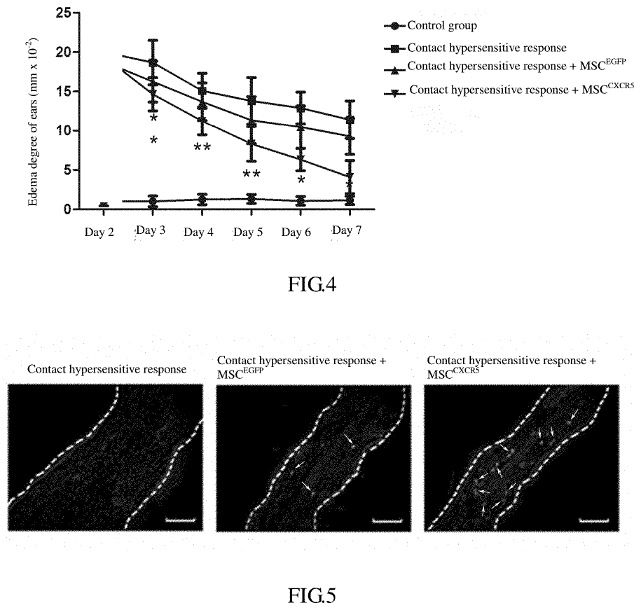

3. The thicknesses of mouse ears on the d1, d2, d3, d4 and d5 after treatment were recorded, the outer edge thicknesses of mouse ears were measured with a micrometer, measurement was carried out three times by the same operator at the same time every day, and an average value was calculated. The results show that in FIG. 4, the thicknesses of mice ears in CHS+MSC^{CXCR5} treatment group are decreased the fastest and the treatment effect is the best. The treatment effect of CHS+MSC^{EGFP} group is between CHS group and CHS+MSC^{CXCR5} treatment group.

4. The quantity of MSC in mouse ears was observed by section staining

Frozen section: the mice were killed by cutting their necks. The ears of each experimental group were taken and immobilized for 6 hours with paraformaldehyde whose volume was 10 times of the ear volume, then transferred to 30% sucrose for dehydration and then overnight at 4° C. The ears were immobilized by OCT and sliced by 7 μm at −20° C. with a frozen slicer;

Immunofluorescence staining: the section was dried for 30 min at 60° C., OCT was removed, the section was washed for 3 times with 0.01 M PBS for 5 minutes, the organized parts were stroked with an immunohistochemistry pen, goat serum was dropwise added, sealed for 30 minutes, primary antibody was added, incubated for overnight at 4° C., placed at room temperature for 30 minutes and washed 3 times with 0.01 M PBS for 5 minutes, the second antibody was added for 30 min and eluted 3 times with 0.01 M PBS for 5 minutes, and DAPI was added for 10 minutes and eluted 3 times with 0.01 M PBS for 5 minutes.

It can be seen from FIG. 5 that after treatment, the thickness of ears in MSC^{CXCR5} treatment group is significantly lower than that of MSC^{EGFP} treatment group, and more importantly, the quantity of MSC in the ears in MSC^{CXCR5} treatment group is significantly higher than that of MSC^{EGFP} treatment group, which proves that targeted migration of MSC^{CXCR5} to inflammatory sites can be realized.

5. Detection of infiltration-myeloperoxidase (MPO) in local inflammatory cells Myeloperoxidase, also known as peroxidase, is a heme protease of a heme cofactor and one of the members of heme peroxidase superfamily. Myeloperoxidase is unique to neutrophils, and is rarely or completely absent in macrophages with strong phagocytosis. In cytochemistry, myeloperoxidase is generally used as a marker for neutrophils. The amount of enzyme in each cell is definite, and accounts for about 5% of the dry weight of cells. This enzyme has the ability of reducing hydrogen peroxide. By utilizing this feature, the activity of enzyme can be analyzed and the number of neutrophils can be quantitatively determined.

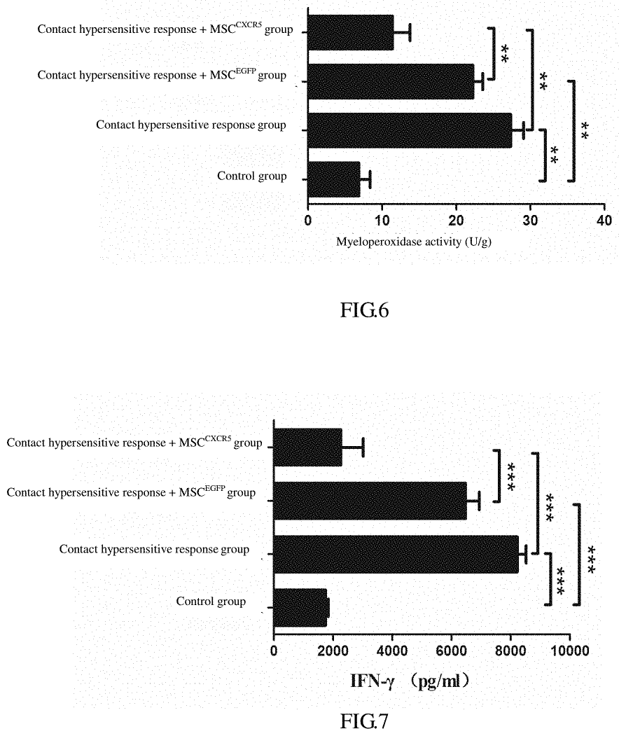

MPO detection: the samples to be detected-the ears of each group of mice were prepared and weighed, the corresponding reagent in the kit was used as a homogenizing medium, the homogenizing medium in a weight/volume ratio of 1:19 was added to be prepared into 5% tissue homogenate, and then the MPO activity was detected according to the steps of the kit. Results are as shown in FIG. 6, the MPO activity of CHS+MSC^{CXCR5} treatment group is significantly lower than that of CHS+MSC^{EGFP} treatment group and CHS group.

The results show that the infiltration of neutrophils in MSC^{CXCR5} treatment group is obviously lower than that in MSC^{EGFP} treatment group, which proves that targeted migration of MSC^{CXCR5} to inflammatory sites can be allowed, MSC^{CXCR5} can exert the immune function and reduces the inflammatory infiltration.

6. Secretion of local inflammatory factors

ELISA detection: 96-well ELISA plate, wash twice with 1×washing buffer, add standard samples and samples to be tested, 100 ul/well, incubate in dark room temperature for 2 h, discard liquid, wash five times with 1×washing buffer, add 100 ul/well of detection antibody incubate in dark room temperature for h, discard liquid, wash five times with 1×washing buffer, remove excess antibody, add 100 ul/well of enzyme conjugate working solution, incubate in dark at room temperature for 30 min, discard the liquid, wash with 1×washing buffer for 5 times, add 5 ul/well of the display substrate, incubate in dark for 20 min, add 5 ul/well of the termination solution, evenly mix, and then detect the OD value of 450 nm by enzyme analyzer.

See FIG. 7, the results show that the infiltration of TNF α and IFN γ in the local inflammatory factors of CHS+MSC^{CXCR5} treatment group is significantly lower than those of CHS+MSC^{EGFP} treatment group and CHS group, which proves that targeted migration of MSC^{CXCR5} to inflammatory sites can be allowed, and MSC^{CXCR5} can exert the immune function and reduces the inflammatory response.

Example 3: TNBS-Induced Mouse Inflammatory BowelDISEASEDisease Model

TNBS (2,4,6-trinitrobenzenesulfonic acid)-induced mouse inflammatory bowel disease model was constructed as follows:

1. Preparation of IBD animal:

(1) Selection of mouse species: BALB/c;

(2) age of mouse: 6 weeks;

(3) Shave 2 cm² in the middle of the right back;

(4) Mice were labeled with picric acid.

2. Preparation of reagent:

(1) Acetone and olive oil were prepared at the ratio of 4:1 to form a mixed solution;

(2) 2,4,6-trinitrobenzene sulfonic acid (TNBS) was dissolved into the mixed solution to prepare the sensitizer;

(3) 4% chloral hydrate.

3. Construction of IBD model:

(1) Mice were slightly anesthetized with 4% chloral hydrate;

(2) a 2×2 cm area was formed on the back with an electric razor, and 150 μl of TNBS pre-sensitization mixed solution was applied;

(3) The mice were conventionally fed for 6 days, and subjected to fasting on the 7^{th} day.

(4) On the d8, mice were slightly anesthetized with 4% chloral hydrate and weighed. A silicone tube with a diameter of about 2 mm was slowly inserted into the anus of mice. When entering 4 cm, 100 μl of TNBS mixed solution was slowly pushed with a syringe (note: venting before operation). The control group was infused with 100 μl of 50% ethanol was injected for control group with the same method. The silicone tube was slowly taken out, the mouse lifted upside down for about 1 minute, so that the sensitization solution slowly entered the colon.

(5) The states of mice were observed and the mice were fed regularly.

(6) The indexes such as symptoms and body weights of mice were observed every day.

4. The animals were divided into 4 groups, 8 mice in each group

\textcircled{1} Control group;

\textcircled{2} Model (CHS) group;

\textcircled{3} MSC^{EGFP} treatment (CHS+MSC^{EGFP}) group;

\textcircled{4} MSC^{CXCR5} treatment (CHS+MSC^{CXCR5}) group.

On the second day after modeling, MSC^{EGFP} was injected intravenously for CHS+MSC^{EGFP} treatment group, 1×10⁶ cells/animal;

MSC^{CXCR5} was injected intravenously for CHS+MSC^{CXCR5} treatment group, 1×10⁶ cells/animal.

The results show that:

1. We detect whether MSC^{CXCR5} chemotaxis to the lesion site can alleviate the inflammatory response of IBD. We infuse MSC^{CXCR5} and MSC^{EGFP} to IBD mice (1×10⁶/mouse) via tail vein during the peak period (day 2), and record the weight of mice every 24 hours, and measure the colon length when the disease is getting better.

The shortening of colon length is a result of generating the reaction of colitis and the leading to edema and contracture. On the d2 after cell infusion, as shown in FIG. 8, the length of colon after MSC^{CXCR5} treatment is significantly longer than those of MSC^{EGFP} group and IBD group, and there is no significant difference between MSC^{EGFP} group and IBD group.

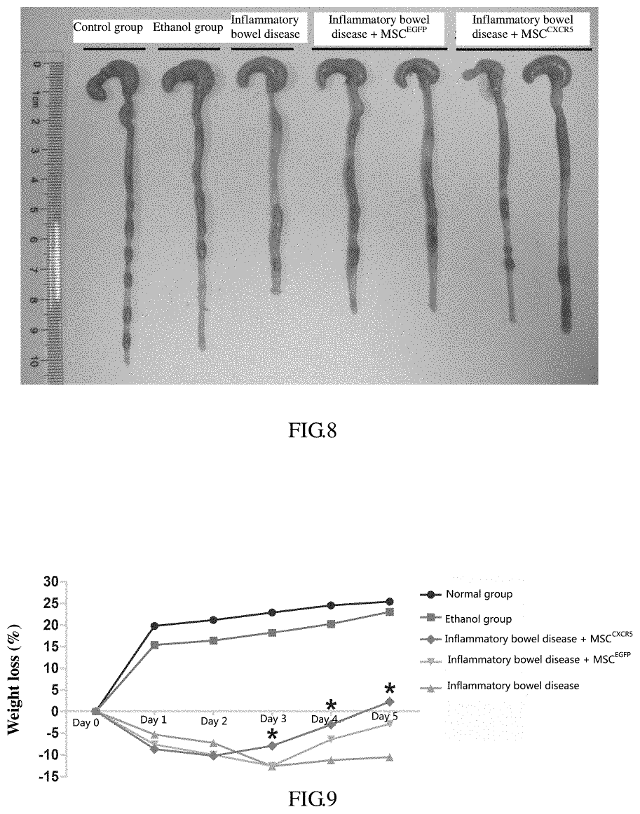

2. The colonic inflammation of mice after MSC^{CXCR5} group treatment is alleviated, specifically, the degree of weight loss was lower than those of MSC^{EGFP} treatment group and IBD group, and the weight is increased after MSC infusion, while the weight recovery of MSC^{EGFP} treatment group is slower, and the weight of IBD group has been in a downward trend after the onset of the disease (FIG. 9).

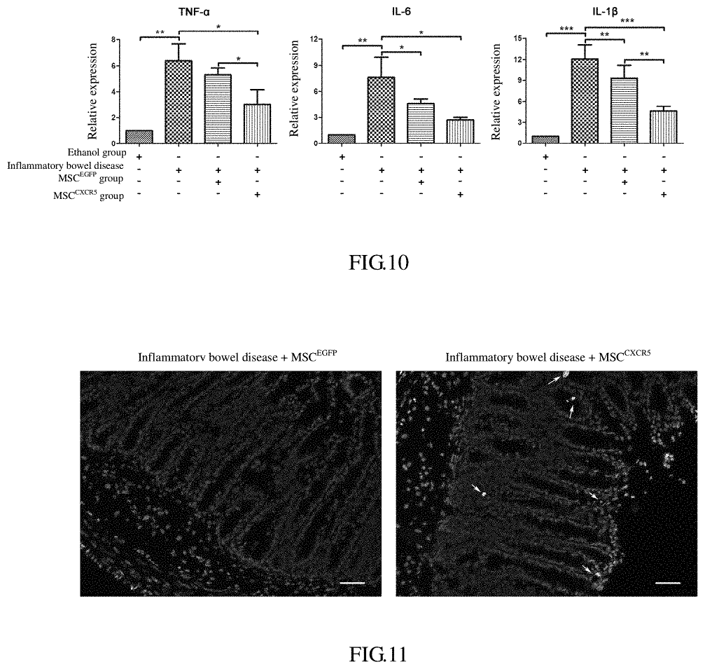

3. To further detect the inflammatory status of IBD lesion tissues, we take the colon tissues of mice in each group and detected the mRNA level of local inflammatory factors. We observe that the levels of proinflammatory factors (TNF-α, IL-6, IL-1 β) in MSC^{CXCR5} treatment group are significantly lower than those in IBD group, while the levels of proinflammatory factors in MSC^{EGFP} treatment group is only slightly lighter than those in IBD group (FIG. 10).

4. The quantity of MSC in mouse colon is observed by section staining

Frozen section: the mice are sacrificed, the colon of each experimental group is taken, immobilized for 6 h with 10-fold volume of paraformaldehyde whose volume and then transferred to 30% sucrose for dehydration, 4° C. overnight and immobilized with OCT, 10 μm of sections are obtained by a freezing microtome at −20° C.;

Immunofluorescence staining: dry tablet, 60° C., 30 minutes, remove OCT, wash PBS of 0.01 M for 5 minutes×3 times, coil the organized part with immunohistochemistry stroke, drop goat serum, seal for 30 minutes, add primary antibody, incubate overnight at 4° C., place at room temperature for 30 minutes, wash with 0.01 M PBS for 5 minutes×3 times, add secondary antibody, 30 minutes, wash with 0.01 M PBS for 5 minutes×3 times, add DAPI for 10 minutes, 0.01 M PBS elution for 5 minutes×3 times, blocking.

From FIG. 11, it can be seen that after treatment, the clear intestinal mucosa layer in MSC^{EGFP} treatment group can be seen, but there is still more cell infiltration in the submucosa; in MSC^{CXCR5} treatment group, the migration focus site of MSC can be seen, the structure of the intestinal mucosa is clear, and the number of inflammatory cell infiltration in the submucosa is few.

Finally, it should be noted that the above examples are only for illustrating the technical solution of the disclosure but not limiting the protective scope of the disclosure. although the disclosure is described in detail with reference to preferred embodiments, those of ordinary skill in the art should be understood that amendments or equivalents can also be made to the technical solution of the disclosure without departing from the spirit and scope of the technical solution of the disclosure.

What is claimed is:

1. Mesenchymal stem cells (MSC) over-expressing CXCR5.

2. A method for treating inflammatory diseases, comprising administering to a subject in need thereof an effective amount of the MSC of claim 1.

3. A method for treating a transplant rejection associated with at least one disease selected from the group consisting of multiple sclerosis, ulcerative colitis, diabetes, bone/cartilage diseases, cardiovascular diseases and nervous system diseases, comprising administering to a subject in need thereof an effective amount of the MSC of claim 1.

4. A drug comprising the MSC of claim 1.

5. A preparation method of the MSC of claim 1, the preparation method comprising: transferring mRNA of CXCR5 into MSC to obtain MSC over-expressing CXCR5.

6. The preparation method of claim 5, wherein the transfer manner is an electroporation transfection method.

7. The preparation method of claim 5, wherein the preparation method of mRNA of CXCR5 comprises the following steps:
(1) collecting and culturing mononuclear cells in peripheral blood from a healthy individual to obtain mononuclear cells of peripheral blood;
(2) extracting RNA of mononuclear cells of peripheral blood;
(3) reverse transcription;
(4) PCR reaction and recovering CXCR5 cDNA fragments;
(5) constructing DNA fragments for transcription of CXCR5 mDNA;
(6) mRNA in vitro transcription.

8. The preparation method of claim 7, wherein primers used in PCR reaction in step (4) are as follows:
upstream primer:
(SEQ ID NO. 1)
ATGAACTACCCGCTAACGCTGG,
downstream primer:
(SEQ ID NO. 2)
CTAGAACGTGGTGAGAGAGGTGGCA.

9. The preparation method of claim 7, wherein the DNA fragment in step (5) is obtained by linking the CXCR5 cDNA fragment in step (4) to plasmids.

10. The preparation method of claim 7, wherein the DNA fragment in step (5) is obtained by performing PCR with the CXCR5 cDNA fragment in step (4) as a template as well as a CXCR5 cDNA upstream primer with a promoter and a CXCR5 cDNA downstream primer as upstream and downstream primers respectively.
